(19) United States
(12) Patent Application Publication
(10) Pub. No.: US 2021/0130396 A1
(43) Pub. Date: May 6, 2021

(54) METHOD FOR PURIFYING PROTEINS

(71) Applicant: ARES TRADING S.A., AUBONNE (CH)

(72) Inventors: HARRY-JAMES SUTTER, VEVEY (CH); XAVIER LE SAOUT, CLARENS (CH); ALBANE FERRARIS, VEVEY (CH)

(21) Appl. No.: 16/640,073

(22) PCT Filed: Aug. 29, 2018
(86) PCT No.: PCT/EP2018/073256

§ 371 Date: Feb. 19, 2020

(30) Foreign Application Priority Data
Aug. 30, 2017 (EP) 17188659.1
Nov. 3, 2017 (EP) 17199869.3

(51) Int. Cl. C07K 1/16; C07K 16/06; C07K 16/28

(52) U.S. Cl. CPC C07K 1/165; C07K 2317/24; C07K 16/28; C07K 16/065

(57) ABSTRACT

The present invention relates to a method for purifying proteins, such as Fc fusion proteins or antibodies, from a sample comprising said proteins and impurities, through the use of a three-chromatographic columns procedure, including a chromatography on hydroxyapatite- and/or Fluorapatite-containing material. The invention is also concerned with pharmaceutical compositions comprising the purified proteins obtainable by the process of the invention.

### FIELD OF THE INVENTION

The present invention relates to a method for purifying proteins, such as Fc fusion proteins or antibodies, from a sample comprising said proteins and impurities, through the use of a three-chromatographic columns procedure, including a chromatography on hydroxyapatite- and/or Fluorapatite-containing material. The invention is also concerned with pharmaceutical compositions comprising the purified proteins obtainable by the process of the invention.

### BACKGROUND OF THE INVENTION

When a protein, such as a fusion protein or an antibody, is produced for therapeutical use, it is important to remove process related impurities, as they may be toxic. Process related impurities typically consist of HCPs (host cell proteins), DNA and rPA (residual protein A). HCPs are an important source of impurity and may represent a serious challenge due to their high complexity and heterogeneity in molecular mass, isoelectric point and structure. It is thus necessary to have therapeutic proteins exhibiting very low levels of HCPs: a particular emphasis should be laid on the optimization of techniques to reduce HCPs during the downstream process (i.e. purification process). Furthermore the downstream process must be tailored in such a way as to comply with the quality produced by the corresponding upstream process. Product related impurities such as aggregates or protein fragments must also be reduced to a minimal level for any kind of therapeutic proteins.

For those willing to produce biosimilars, there is an addition factor to be taken into account: the charge variants. Indeed, the content of acidic and basic charge variants must lay within the biosimilar corridor defined by the Reference Product. Considering that charge variants may be altered by upstream as well as downstream processing, the downstream process must be adapted to this challenge.

Additionally, for any kind of therapeutic proteins, the purification should minimize process related protein loss and target an acceptable yield at each process step.

There is a need to find optimal purification sequence which guarantees the overall clearance of product and process related impurities according to quality criteria, while minimizing protein loss due to the purification process.

### SUMMARY OF THE INVENTION

In one aspect, the present invention provides a method of purifying a protein, such as a Fc fusion protein or an antibody, from a sample containing the protein and impurities, wherein the method comprises the following steps: (a) contacting the sample containing the protein and the impurities with a Protein A chromatography material (either a resin or a membrane) under conditions such that the protein binds to the chromatography material and at least a portion of the impurities does not bind to the chromatography material; (b) eluting the protein from the Protein A chromatography material, in order to obtain an eluate; (c) loading the eluate of step (b) onto a first mixed mode chromatography material (either a resin or a membrane) under conditions such that the protein does not bind to the chromatography material and at least a portion of the remaining impurities binds to the chromatography material; (d) recovering the flowthrough containing the protein under conditions such that said recovered flowthrough contains a lower level of impurities than the eluate of step (b), (e) loading the recovered flowthrough containing the protein of step (d) onto a second mixed mode chromatography material (either a resin or a membrane) under conditions such that the protein does not bind to the chromatography material and at least a portion of the remaining impurities binds to the chromatography material; and (f) recovering the flowthrough containing the protein under conditions such that said recovered flowthrough contains a lower level of impurities than the recovered flowthrough of step (d).

In another aspect, the present invention also provides a method of obtaining a protein in a monomeric form, wherein the method comprises the following steps: (a) contacting the sample containing the protein in monomeric form, aggregated forms or fragmented forms with a Protein A chromatography material (either a resin or a membrane) under conditions such that the protein in monomeric form binds to the chromatography material and at least a portion of the aggregated forms and fragmented forms does not bind to the chromatography material; (b) eluting the protein in monomeric form from the Protein A chromatography material, in order to obtain an eluate; (c) loading the eluate of step (b) onto a first mixed mode chromatography material (either a resin or a membrane) under conditions such that the protein in monomeric form does not bind to the chromatography material and at least a portion of the remaining aggregated forms and fragmented forms bind to the chromatography material; (d) recovering the flowthrough containing the protein in monomeric form under conditions such that said recovered flowthrough contains a lower level of aggregated forms and fragmented forms than the eluate of step (b), (e) loading the recovered flowthrough containing the protein in monomeric form of step (d) onto a second mixed mode chromatography material (either a resin or a membrane) under conditions such that the protein in monomeric form does not bind to the chromatography material and at least a portion of the remaining aggregated forms and fragmented forms bind to the chromatography material; and (f) recovering the flowthrough containing the protein in monomeric form under conditions such that said recovered flowthrough contains a lower level of aggregated forms and fragmented forms than the recovered flowthrough of step (d).

The protein to be purified (also referred to as the protein of interest) according to the present invention can be an Fc fusion protein (also referred to as the Fc fusion protein of interest) or an antibody (also referred to as the antibody of interest). The Fc fusion protein preferably comprises either an Fc portion or is a fusion protein based on an antibody moiety. An antibody of interest can be a chimeric antibody, a humanized antibody or a fully human antibody, or other kind of antibody such as SEEDbody.

The mixed mode chromatography material (also referred to as chromatography support) of the present invention can be under the form of resins or membranes and present a combination of two or more of the following functionalities such as cation exchange, anion exchange, hydrophobic interaction, hydrophilic interaction, hydrogen bonding. Preferably, the mixed mode chromatography support for step (c) is for instance selected from the group consisting of Capto-MMC or Capto-Adhere and the mixed mode chromatography support of step (e) is selected from the group consisting of hydroxyapatite and/or fluorapatite.

### DEFINITION

The term “antibody”, and its plural form “antibodies”, includes, inter alia, polyclonal antibodies, affinity-purified polyclonal antibodies, monoclonal antibodies, and antigen-binding fragments. Antibodies are also known as immunoglobulins. Genetically engineered intact antibodies or fragments, such as chimeric antibodies, humanised antibodies, human or fully human antibodies, as well as synthetic antigen-binding peptides and polypeptides, are also included. Also encompassed are SEEDbodies. The term SEEDbody (SEED for Strand-Exchange Engineered Domain; plural form: SEEDbodies), refers to a particular type of antibody comprising derivative of human IgG and IgA CH3 domains, creating complementary human SEED CH3 heterodimers that are composed of alternating segments of human IgG and IgA CH3 sequences. They are asymmetric fusion proteins. SEEDbodies and the SEED technology are described in Davis et al. 2010 ([1] or U.S. Pat. No. 8,871,912 ([2]) which are incorporated herein in their entirety.

The term “monoclonal antibody” refers to an antibody that is a clone of a unique parent cell. The term “humanized” immunoglobulin (or “humanized antibody”) refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor” (humanization by grafting non-human CDRs onto human framework and constant regions, or by incorporating the entire non-human variable domains onto human constant regions (chimerization)). Constant regions need not be present in their entirety, but if they are, they must be substantially identical to human immunoglobulin constant regions, i.e., at least about 85-90%, preferably about 95% or more identical. Hence, all parts of a humanized immunoglobulin, except possibly the CDRs and a few residues in the heavy chain constant region if modulation of the effector functions is needed, are substantially identical to corresponding parts of natural human immunoglobulin sequences. Through humanizing antibodies, biological half-life may be increased, and the potential for adverse immune reactions upon administration to humans is reduced.

The term “fully human” immunoglobulin (or “fully-human” antibody) refers to an immunoglobulin comprising both a human framework region and human CDRs. Constant regions need not be present in their entirety, but if they are, they must be substantially identical to human immunoglobulin constant regions, i.e., at least about 85-90%, preferably about 95% or more identical. Hence, all parts of a fully human immunoglobulin, except possibly few residues in the heavy chain constant region if modulation of the effector functions or pharmacokinetic properties are needed, are substantially identical to corresponding parts of natural human immunoglobulin sequences. In some instances, amino acid mutations may be introduced within the CDRs, the framework regions or the constant region, in order to improve the binding affinity and/or to reduce the immunogenicity and/or to improve the biochemical/biophysical properties of the antibody.

The term “recombinant antibody” (or “recombinant immunoglobulin) means antibody produced by recombinant technics. Because of the relevance of recombinant DNA techniques in the generation of antibodies, one needs not be confined to the sequences of amino acids found in natural antibodies; antibodies can be redesigned to obtain desired characteristics. The possible variations are many and range from the changing of just one or a few amino acids to the complete redesign of, for example, the variable domain or constant region. Changes in the constant region will, in general, be made in order to improve, reduce or alter characteristics, such as complement fixation (e.g. complement dependent cytotoxicity, CDC), interaction with Fc receptors, and other effector functions (e.g. antibody dependent cellular cytotoxicity, ADCC), pharmacokinetic properties (e.g. binding to the neonatal Fc receptor; FcRn). Changes in the variable domain will be made in order to improve the antigen binding characteristics. In addition to antibodies, immunoglobulins may exist in a variety of other forms including, as well as diabodies, linear antibodies, multivalent or multispecific hybrid antibodies.

The terms “monomeric form”, “aggregated form” and “fragmented form” are to be understood as per the common general knowledge. Therefore, the terms “monomeric form” refers to an Fc fusion protein or an antibody not associated with a second similar molecule, the term “aggregated form” (also called High Molecular weight species; HMW) refers to an Fc fusion protein or an antibody which is associated, either covalently or non-covalently with a second similar molecule and the term “fragmented form” (also called Low Molecular weight species; LMW) relates to single parts of Fc fusion protein or an antibody (e.g. light and/or heavy chains). “Monomeric form” does not mean that the protein (such as Fc fusion protein or an antibody) is 100% in monomeric form, but simply essentially in monomeric form, i.e. at least 95% in monomeric form, or preferably 97% in monomeric form or even more preferably at least 98% in monomeric form. As there is a balance between monomeric forms, aggregates forms and fragmented forms (total amount of the 3 species=100%), when aggregates and fragmented forms are reduced, monomeric forms are increased.

The “total purification factor” refers to the “total reduction factor” for the species that is analysed, leading to a better purification of the protein of interest (e.g. in the monomeric form). The higher total purification factor, the better.

The term “Fc fusion protein” encompasses the combination (also called fusion) of at least two proteins or at least two proteins fragments to obtain one single protein, including either an Fc portion or an antibody moiety.

The term “buffer” is used according to the art. An “equilibration buffer” is a buffer used to prepare the chromatography material to receive the sample to be purified. A “loading buffer” refers to the buffer used to load the sample on the chromatography material or on a filter. A “wash buffer” is a buffer used to wash the resin. Depending on the mode of the chromatography it will allow the removal of the impurities (in bind/elute mode) or the collection of the purified sample (in flowthrough mode). An “elution buffer” refers to the buffer that is used to unbind the sample from the chromatographic material. This is possible thanks to the change of ionic strength between the load/wash buffers and the elution buffer. The purified sample containing the antibody will thus be collected as an eluate. The term “chromatographic material” or “chromatography material” (also referred to as chromatographic support or chromatography support) such as “resin” or “membrane” refer to any solid phase/membrane allowing the separation of the molecule to be purified from the impurities. Said resin, membrane or chromatographic material may be an affinity, an anionic, a cationic, an hydrophobic or a mixed mode resin/chromatographic material.

Examples of known antibodies which can be produced according to the present invention include, but are not limited to, adalimumab, alemtuzumab, atezolizumab, avelumab, belimumab, bevacizumab, canakinumab, certolizumab pegol, cetuximab, denosumab, eculizumab, golimumab, infliximab, natalizumab, nivolumab, ofatumumab, omalizumab, pembrolizumab, pertuzumab, pidilizumab ranibizumab, rituximab, siltuximab, tocilizumab, trastuzumab, ustekinumab or vedolizomab.

Units, prefixes and symbols are used according to the standards (International System of Units (SI)).

### DETAILED DESCRIPTION OF THE INVENTION

### A. General

It was found by the inventors that using the sequence “Protein A chromatography” followed by a first “mixed mode chromatography” in flowthrough followed by a second “mixed mode chromatography” also in flowthrough allows among other to reduce, in a sample of proteins, the amount of impurities, such as aggregates and low molecular weight species, while keeping HCPs in acceptable ranges. The sample of proteins (such as antibodies or Fc fusion proteins) to be purified according to the process of the present invention is preferably obtained at the time of harvest or post-harvest, should the sample be hold for a certain amount of time before purification.

Therefore, in a first aspect, the present invention provides a method of purifying a protein from a sample containing the protein and impurities, wherein the method comprises the following steps: (a) contacting the sample containing the protein and the impurities with an affinity chromatography material (either a resin or a membrane) under conditions such that the protein binds to the chromatography material and at least a portion of the impurities does not bind to the chromatography material; (b) eluting the protein from the affinity chromatography material, in order to obtain an eluate; (c) loading the eluate of step (b) onto a first mixed mode chromatography material (either a resin or a membrane) under conditions such that the protein does not bind to the chromatography material and at least a portion of the remaining impurities binds to the chromatography material; (d) recovering the flowthrough containing the protein under conditions such that said recovered flowthrough contains a lower level of impurities than the eluate of step (b), (e) loading the recovered flowthrough containing the protein of step (d) onto a second mixed mode chromatography material (either a resin or a membrane) under conditions such that the protein does not bind to the chromatography material and at least a portion of the remaining impurities binds to the chromatography material; and (f) recovering the flowthrough containing the protein under conditions such that said recovered flowthrough contains a lower level of impurities than the recovered flowthrough of step (d).

In a second aspect, the present invention describes a method of obtaining a protein in a monomeric form, wherein the method comprises the following steps: (a) contacting the sample containing the protein in monomeric form, aggregated form or fragmented form with an affinity chromatography material (either a resin or a membrane) under conditions such that the protein binds to the chromatography material and at least a portion of the aggregated form and fragmented form does not bind to the chromatography material; (b) eluting the protein in monomeric form from the affinity chromatography material, in order to obtain an eluate; (c) loading the eluate of step (b) onto a first mixed mode chromatography material (either a resin or a membrane) under conditions such that the protein in monomeric form does not bind to the chromatography material and at least a portion of the remaining aggregated form and fragmented form bind to the chromatography material; (d) recovering the flowthrough containing the protein in monomeric form under conditions such that said recovered flowthrough contains a lower level of aggregated form and fragmented form than the eluate of step (b), (e) loading the recovered flowthrough containing the protein in monomeric form of step (d) onto a second mixed mode chromatography material (either a resin or a membrane) under conditions such that the protein in monomeric form does not bind to the chromatography material and at least a portion of the remaining aggregated form and fragmented form bind to the chromatography material; and (f) recovering the flowthrough containing the protein in monomeric form under conditions such that said recovered flowthrough contains a lower level of aggregated form and fragmented form than the recovered flowthrough of step (d).

In the context of the present invention as a whole, the impurities to be removed are preferably selected from the group comprising or consisting of aggregates of the protein of interest or fragments of said protein of interest or mixtures thereof, one or more of host cell proteins, endotoxins, viruses, nucleic acid molecules, lipids, polysaccharides, and any combinations thereof. The protein to be purified according to the present invention can be any kind of antibodies, such as monoclonal antibodies, or Fc fusion proteins. When the protein of interest is an Fc fusion protein, it comprises either an Fc portion or is derived from an antibody moiety or from an antibody fragment and contained at least CH2/Ch3 domains of said antibody moiety or fragment. When the protein of interest is a monoclonal antibody it can be a chimeric antibody, a humanized antibody or a fully human antibody or any fragment thereof. The protein of interest to be purified can first be produced in a prokaryotic or eukaryotic cell, such as a bacterium, a yeast cell, insect cell or a mammalian cell. Preferably, the protein of interest has been produced in recombinant mammalian cells. Said mammalian host cell (herein also refer to as a mammalian cell) includes, but not limited to, HeLa, Cos, 3T3, myeloma cell lines (for instance NS0, SP2/0), and Chinese hamster ovary (CHO) cells. In a preferred embodiment, the host cell is a Chinese Hamster Ovary (CHO) cell, such as such as CHO-S cell and CHO-k1 cell. The cell lines (also referred to as “recombinant cells” or “host cells”) used in the invention are genetically engineered to express the protein of interest. Methods and vectors for genetically engineering of cells and/or cell lines to express the polypeptide of interest are well known to those of skill in the art; for example, various techniques are illustrated in Sambrook et al. ([3]) or Ausubel et al. ([4]). The protein of interest produced according to said methods is called a recombinant protein. The recombinant proteins are usually secreted into the culture medium from which they can be recovered. The recovered proteins can then be purified, or partially purified using known processes and products available from commercial vendors. The purified proteins can be formulated as pharmaceutical compositions. Suitable formulations for pharmaceutical compositions include those described in Remington's Pharmaceutical Sciences (1995 and updated; [5]).

Typically, the methods according to the invention are performed at room temperature (between 15° C. and 25° C.), except for the loading of step (a) typically performed/started between 2 to 8° C. as the sample containing the protein to be purified is usually stored in cold conditions (typically between 2 to 8° C.) after harvest as per standard procedures (see [6]).

The recovered sample of step f), comprising the purified antibody, comprises preferably aggregates at a level of at least 50% lower than the level of aggregates in the sample of step (a), preferably at a level of at least 60% lower than the level of aggregates in the sample of step (a), even preferably at a level of at least 70% lower than the level of aggregates in the sample of step (a), and even preferably at a level of at least 80% lower than the level of aggregates in the sample of step (a). Similarly, said recovered sample comprises preferably fragments at a level of at least 10% lower than the level of fragments in the sample of step (a) or even preferably fragments at a level of at least 20% lower than the level of fragments in the sample of step (a). HCPs are comprised at a level preferably below the typical acceptable limit of 100 ppm.

Preferably, the purification method described herein does not comprises more than three chromatographic steps. More preferably, the purification method described herein consists of only three chromatographic steps (i.e. an affinity chromatography step and two mixed mode chromatography steps), optionally comprising filtration steps and/or other virus inactivation steps.

Even more preferably, the purification method described herein consists of only three chromatographic steps performed according to specific mode: i.e. an affinity chromatography step in bind/elute mode and two mixed mode chromatography steps in flow-through mode, optionally comprising filtration steps and/or other virus inactivation steps.

The purification method described herein can be performed “stepwise” or in continuous mode for a part or all of the steps.

### B. Affinity Chromatography Step (Steps (a) and (b))

B.1. General

The term “Protein A chromatography” refers to the affinity chromatography technic using protein A, in which the protein A is usually immobilized on a solid phase. Protein A is a surface protein originally found in the cell wall of the bacteria Staphylococcus aureus. It now exists various kind of protein A of natural original or produced recombinantly, possibly comprising some mutations as well. This protein has the ability to specifically bind the Fc portion of immunoglobulin such as IgG antibodies or any Fc fusion proteins.

Protein A chromatography is one of the most common affinity chromatography used for purifying antibodies and Fc fusion proteins. Typically, the antibodies (or Fc fusion proteins) from a solution to be purified reversibly bind to the protein A, via their Fc portion. To the contrary (most of) the impurities flow through the column and are eliminated via washing steps. The antibodies (or Fc fusion proteins) thus need to be eluted from the column, or the affinity resin, in order to be collected for the next purification steps.

The protein A chromatography material in step (a) in the context of the present invention as a whole is selected for instance from the group consisting of, but not limited to, MABSELECT™, MABSELECT™ SuRe, MABSELECT™ SuRe LX, AMSPHERE™ A3, TOYOPEARL ® AF-rProtein A-650F, TOYOPEARL® AF-HC, PROSEP®-vA, PROSEP®-vA Ultra, PROSEP® Ultra Plus or ESHMUNO-A® and any combination thereof. In some embodiments, the Protein A ligand is immobilized on a resin selected from the group consisting of dextran based matrix, agarose based matrix, polystyrene based matrix, hydrophilic polyvinyl ethyl based matrix, rigid polymethacrylate based matrix, porous polymer based matrix, controlled pore glass based matrix, and any combination thereof. Alternatively, the Protein A ligand is immobilized on a membrane. The purpose of this step is to capture the protein of interest present in the clarified harvest, concentrate them and remove most of the process-related impurities (e.g. HCPs, DNA, components of the cell culture broth).

B.2. Equilibration and Loading

In the context of the present invention as a whole, the sample, containing the protein of interest, to be contacting with the affinity chromatography material in step (a) is in an aqueous solution. It can be a crude harvest, a clarified harvest or even a sample pre-equilibrated in an aqueous buffered solution.

Before purification of the sample, the Protein A material has to be equilibrated. This equilibration is performed with an aqueous buffered solution. Suitable aqueous buffered solution (or buffers) include, but are not limited to, phosphate buffers, Tris buffers, acetate buffers, and/or citrate buffers. The aqueous buffered solution for this step is preferably based on sodium acetate or sodium phosphate. Preferably, the buffered solution is at a concentration in the range of or of about 10 mM to or to about 40 mM and a pH in the range of or of about 6.5 to or to about 8.0. Even preferably, the buffered solution is at a concentration in the range of or of about 15 mM to or to about 30 mM and its pH in the range of or of about 6.8 to or to about 7.5. Even preferably, the concentration of the buffered solution is at or at about 15.0, 16.0, 17.0, 17.0, 18.0, 19.0, 20.0, 21.0, 22.0, 23.0, 24.0 or 25.0 mM and its pH is at or at about 6.5, 6.6, 6.7, 6.8, 6.9, 7.0, 7.1, 7.2, 7.3, 7.4 and 7.5.

The aqueous buffered solution to be used in one of the methods according to the invention can further comprise a salt at a concentration in the range of or of about 100 mM to or to about 200 mM, preferably at a concentration in the range of or of about 125 to 180 mM, such as of or of about 130, 135, 140, 145, 150, 155, 160, 165, or 170 mM. Suitable salts include, but are not limited to, sodium chloride.

The skilled person will choose the appropriate conditions for equilibration and loading in order that the protein to be purified does bind to the affinity chromatography material. To the contrary, at least a part of the impurities will flow through the chromatography material. For instance, the aqueous buffered solution for equilibration comprises sodium phosphate at or at about 25 mM and a pH at 7.0±0.2 and sodium chloride at a concentration of or of about 150 mM.

B.3. Washing

After loading (step (a)), the affinity chromatography material is washed once or twice, with more of the same solution as the equilibration buffer or a different one, or a combination of both. As for the equilibration and loading step, suitable aqueous buffered solution (or buffers) include, but are not limited to, phosphate buffers, Tris buffers, acetate buffers, and/or citrate buffers. The wash step is necessary to remove the unbound impurities.

Preferably, the wash is performed in one step, i.e. with one buffer. Preferably the wash buffer is an acetate buffer (such as a sodium acetate buffer) at a concentration in the range of or of about 40 mM to or to about 70 mM and a pH in the range of or of about 5.0 to or to about 6.0. Even preferably, the buffered solution is at a concentration in the range of or of about 45 mM to or to about 65 mM and its pH in the range of or of about 5.2 to or to about 5.8. Even preferably, the concentration of the buffered solution is at or at about 50, 51, 52, 53, 54, 55, 56, 57, 58, 59 or 60 mM and its pH is at or at about 5.2, 5.3, 5.4, 5.5, 5.6, 5.7 and 5.8.

In an alternative, the wash is performed in two steps with two different buffers. Preferably the first wash buffer is an acetate buffer (such as a sodium acetate buffer) at a concentration in the range of or of about 40 mM to or to about 70 mM and a pH in the range of or of about 5.0 to or to about 6.0. Even preferably, the buffered solution is at a concentration in the range of or of about 45 mM to or to about 65 mM and its pH in the range of or of about 5.2 to or to about 5.8. Even preferably, the concentration of the buffered solution is at or at about 50, 51, 52, 53, 54, 55, 56, 57, 58, 59 or 60 mM and its pH is at or at about 5.2, 5.3, 5.4, 5.5, 5.6, 5.7 and 5.8. Preferably, the second wash buffer is similar to the equilibration/loading buffer.

The aqueous buffered solution to be used in one of the methods according to the invention can further comprises a salt. Preferably, should a salt be present and should the method comprise a two-wash-step, said salt will be in a higher concentration in the first wash buffer than in the second wash buffer. Preferably, the concentration of salt in the wash buffer (when 1-step only) or in the first wash buffer (when 2-steps), if any, is at a concentration in the range of or of about 1.0 M to or to about 2.0 M, preferably at a concentration in the range of or of about 1.25 to 1.80 M, such as of or of about 1.3, 1.3.5, 1.4, 1.45, 1.5, 1.55, 1.6, 1.65, or 1.70 M. Preferably, the concentration of salt in the second wash buffer, if any, is at a concentration in the range of or of about 100 mM to or to about 200 mM, preferably at a concentration in the range of or of about 125 to 180 mM, such as of or of about 130, 135, 140, 145, 150, 155, 160, 165, or 170 mM. Suitable salts include, but are not limited to, sodium chloride, potassium chloride, ammonium chloride, sodium acetate, potassium acetate, ammonium acetate, calcium salts, and/or magnesium salts.

The skilled person will chose the appropriate conditions for washing step in order that the protein to be purified remain bound to the affinity chromatography material. To the contrary, at least a part of the impurities will continue to flow through the chromatography material thanks to the wash buffers. As a non-limiting example, with a 2-steps wash, if the equilibration buffer comprises sodium phosphate at or at about 25 mM, a salt at a concentration of or of about 150 mM and has a pH at 7.0±0.2, a first wash can be performed with a wash buffer comprising phosphate at or at about 55 mM, a salt at a concentration of or of about 1.5 M and a pH of 5.5±0.2 and a second wash can be performed with a wash buffer identical to the equilibration buffer.

B.4. Elution

The protein of interest can then be eluted (step (b)) using a solution (called elution buffer) that interferes with the binding of the affinity chromatography material to the Fc moiety/constant domain of the protein to be purified. This elution buffer may include acetic acid, glycine, citrate or citric acid.

Preferably, the buffered solution is an acetic acid buffer at a concentration in the range of or of about 40 mM to or to about 70 mM. Even preferably, the buffered solution is at a concentration in the range of or of about 45 mM to or to about 65 mM. Even preferably, the concentration of the buffered solution is at or at about 50, 51, 52, 53, 54, 55, 56, 57, 58, 59 or 60 mM. Elution may be performed by lowering the pH of the chromatography material and proteins attached thereto. For example, the pH of the elution buffer can be at or at about 4.5 or less, or at or at about 4.0 or less. It is preferably at or at about 2.8 to or to about 3.7, such as 2.9, 3.0, 3.1, 3.2, 3.3, 3.4, 3.5 or 3.6. The elution buffer optionally include a chaotropic agent.

The skilled person will choose the appropriate conditions for elution step in order that the protein to be purified is released from the affinity chromatography material. As a non-limiting example, the elution (i.e. elution of step (b)) can be performed with an elution buffer comprising acetic acid at or at about 55 mM and a pH of 3.2±0.2.

### C. Mixed Mode Chromatography Steps

C.1. General

The mixed mode chromatography material (also referred to as mixed mode chromatography support) according to the present invention refers to a chromatographic material that involves a combination of two or more of the following functionalities (but not limited to): cation exchange, anion exchange, hydrophobic interaction, hydrophilic interaction, hydrogen bonding or metal affinity. It thus comprises two different types of ligands. The solid phase can be a matrix such as a resin, porous particle, nonporous particle, membrane, or monolith.

C.2. First Mixed Mode Chromatography (steps (c) and (d))

In the context of the present invention as a whole, the preferred mixed mode chromatography support for step (c) is selected from the group consisting of Capto-MMC, Capto-Adhere, Capto adhere Impress, MEP Hypercel and ESHMUNO HCX. It is preferably a support having anion exchange properties such as Capto-Adhere. Alternatively, the mixed mode chromatography material can be a membrane such as the Natrix HD-SB.

Preferably, before being loaded the eluate recovered after affinity chromatography (i.e. eluate of step (b)) is adjusted to a pH of 6.5 to 8.5 such as 6.7, 6.8, 6.9, 7.0, 7.1, 7.2, 7.3, 7.4, 7.5, 7.6, 7.7, 7.8, 7.9, 8.0, 8.1 or 8.2. pH adjustment can be done with a concentrated solution of TRIS and/or NaOH for instance. The aim is to have the eluate of step (b) at a pH and conductivity similar to the one under which step (c) is to be performed. Said eluate will thus be an adjusted eluate. If step (c) for instance is to be performed at a pH of 8.0±0.2, the eluate of step (b) has to be adjusted to a pH of 8.0±0.2. Similarly if step (c) is to be performed with a salt, same salt conditions will be used for the adjustment.

Before being loaded with the adjusted eluate, the first mixed mode chromatography material is equilibrated with an aqueous buffered solution (equilibration buffer). Suitable aqueous buffered solution (or buffers) include, but are not limited to, phosphate buffers, Tris buffers, acetate buffers, and/or citrate buffers. Preferably, the buffered solution, e.g. a sodium phosphate buffer, is at a concentration in the range of or of about 20 mM to or to about 60 mM and a pH in the range of or of about 6.5 to or to about 8.5. Even preferably, the buffered solution is at a concentration in the range of or of about 30 mM to or to about 50 mM and its pH in the range of or of about 6.5 to or to about 8.5. Even preferably, the concentration of the buffered solution is at or at about 35, 36, 37, 38, 39, 40, 41, 42, 43, 44 or 45 mM and its pH is at or at about 6.8, 6.9, 7.0, 7.1, 7.2, 7.3, 7.4, 7.5, 7.6, 7.7, 7.8, 7.9, 8.0, 8.1 or 8.2.

The aqueous buffered solution to be used in one of the methods according to the invention can further comprises a salt at a concentration in the range of or of about 50 mM to or to about 1 M, preferably at a concentration in the range of or of about 85 to 500 mM, such as of or of about 100, 150, 200, 250, 300, 350, 400, 450 or 500 mM. Suitable salts include, but are not limited to, sodium chloride and/or potassium chloride.

The equilibration buffer will also be used to “push” the unbound protein of interest in the flowtrough, in order to recover said purified antibodies/proteins (step d). Said flowthrough is recovered at the bottom of the column. To the contrary, at least a part of the impurities binds to the chromatography material.

Once the mixed mode chromatography material is equilibrated, the eluate of step (b) (or the adjusted eluate) can be loaded. The unbund protein of interest will be pushed by the addition of equilibration buffer and recovered at the bottom of the column.

In the context of the invention, the skilled person will choose the appropriate conditions for this first mixed mode chromatography step in order that the protein to be purified does not bind to the first mixed mode chromatography material, i.e. in order that it flows through the chromatography material. The skilled person knows how to adapt the pH and/or the salt condition of the buffer in view of the pI (Isoelectric Point) of the protein to be purified. As a non-limiting example, e.g. for an protein of interest having a pI above 9.0, the equilibration buffer for the first mixed mode chromatography step can comprise sodium phosphate at or at about 40 mM, a sodium chloride at a concentration of about 95 mM and a pH of 8.0±0.2. Loading is performed in the same condition. As a further non- limiting example, e.g. for an protein of interest having a pI about 8.5 to about 9.5, the equilibration buffer for the first mixed mode chromatography step can comprise sodium phosphate at or at about 40 mM, a sodium chloride at a concentration of or of about 470 mM and a pH of or of about 7.3±0.2. Loading is performed in the same condition.

C.3. Second Mixed Mode Chromatography (Steps (e) and (f)

In the context of the present invention as a whole, the preferred mixed mode chromatography support for the second mixed mode chromatography step (step (e)) comprises ligand(s) selected from the group consisting of hydroxy-based ligand and/or fluorapatite-based ligand. Such ligands can be used for instance in chromatographic material such as resin or membrane.

An hydroxyapatite-based ligand comprises a mineral of calcium phosphate with the structural formula (Ca₅(PO₄)₃OH)₂. Its dominant modes of interaction are phosphoryl cation exchange and calcium metal affinity. Mixed mode chromatography supports comprising said hydroxyapatite-based ligand are commercially available in various forms, including but not limited to ceramic forms. Commercial examples of ceramic hydroxyapatite include, but are not limited to CHT™ Type I and CHT™ Type II. Ceramic hydroxyapatites are porous particles and can have various diameters, for instance about 20, 40, and 80 microns.

A fluorapatite-based ligand comprises an insoluble fluoridated mineral of calcium phosphate with the structural formula Ca₅(PO₄)₃F or Ca₁₀(PO₄)₆F₂. Its dominant modes of interaction are phosphoryl cation exchange and calcium metal affinity. Mixed mode chromatography supports comprising said fluorapatite-based ligand are commercially available in various forms, including but not limited to ceramic forms. Commercial examples of ceramic fluorapatite include, but are not limited to CFT™ Type I and CFT™ Type II. Ceramic fluorapatites are spherical porous particles and can have various diameters, for instance about 10, 20, 40, and 80 microns.

A hydroxyfluorapatite-based ligand comprises an insoluble hydroxylated and an insoluble fluoridated mineral of calcium phosphate with the structural formula Ca10(P04)6(OH)x(F)y. Its dominant modes of interaction are phosphoryl cation exchange and calcium metal affinity. Mixed mode chromatography supports comprising said hydroxyfluoroapatite ligand are commercially available in various forms, including but not limited to ceramic, crystalline and composite forms. Composite forms contain hydroxyfluorapatite microcrystals entrapped within the pores of agarose or other beads. An example of ceramic hydroxyfluorapatite resin is the MPC Ceramic Hydroxyfluorapatite Resin™, with a structural formula (Ca₁₀(PO₄)₆(OH)_{1.5}(F)_{0.5}), It is based on the ceramic apatite Type I (40 μm) mixed-mode resin.

Preferably, before being loaded, the flowthrough recovered after the first mixed mode chromatography (i.e. eluate of step (d)) is adjusted to a pH of 7.0 to 8.5 such as 7.0, 7.1, 7.2, 7.3, 7.4, 7.5, 7.6, 7.7, 7.8, 7.9, 8.0, 8.1, or 8.2. Adjustment can be done with a concentrated solution of TRIS and/or NaOH for instance. Said eluate will thus be an adjusted eluate. The aim is to have the flowthrough of step (d) into conditions suitable for the load on the second mixed mode chromatography. If step (e) for instance is to be performed at a pH of 7.5±0.2, the flowthrough of step (d) has to be adjusted to a pH of 7.5±0.2. This step of adjustment can be performed together with a concentration step. In such a case, a filtration step can be added before the second mixed mode chromatography. Other adjustments that can be needed relate to salts and NaPO₄.

Before being loaded with the adjusted flowthrough containing the protein of interest, the first mixed mode chromatography material is equilibrated with an aqueous buffered solution (equilibration buffer). Preferably, the flowthrough recovered after the first mixed mode chromatography step (step (d)) is equilibrated prior to loading onto the second mixed mode chromatography material (of step (e)) with an aqueous buffered solution. Suitable aqueous buffered solution (or buffers) include, but are not limited to, phosphate buffers, Tris buffers, acetate buffers, and/or citrate buffers. Preferably, the buffered solution, e.g. a sodium phosphate buffer is at a concentration in the range of or of about 1 mM to or to about 20 mM and a pH in the range of or of about 7.0 to or to about 8.5. Even preferably, the buffered solution is at a concentration in the range of or of about 2 mM to or to about 15 mM and its pH in the range of or of about 7.2 to or to about 7.8. Even preferably, the concentration of the buffered solution is at or at about 2.5, 3.0, 3.5, 4.0, 4.5, 5.0, 5.5, 6.0, 6.5, 7.0, 8.0, 9.0, 10.0 mM and its pH is at or at about 7.2, 7.3, 7.4, 7.5, 7.6, 7.7 and 7.8.

The aqueous buffered solution to be used in one of the methods according to the invention can further comprises a salt at a concentration in the range of or of about 50 mM to or to about 1 M, preferably at a concentration in the range of or of about 85 to 500 mM, such as of or of about 100, 150, 200, 250, 300, 350, 400, 450 or 500 mM. Suitable salts include, but are not limited to sodium chloride and/or potassium chloride.

The equilibration buffer will also be used to “push” the unbound protein of interest (e.g. antibodies or Fc fusion proteins) in the flowtrough, in order to recover said purified proteins (step f). Said flowthrough is recovered at the bottom of the column. To the contrary, at least a part of the impurities bind to the chromatography material.

Once the mixed mode chromatography material is equilibrated, the eluate of step (d) (or adjusted eluate) can be loaded. The unbund protein of interest will be pushed by the addition of equilibration buffer and recovered at the bottom of the column.

In the context of the invention, the skilled person will choose the appropriate conditions (in view of the pI of the protein to be purified) for this second mixed mode chromatography step in order that the protein to be purified does not bind to the first mixed mode chromatography material, i.e. in order that it flows through the chromatography material. As a non-limiting example, e.g. for an protein of interest having a pI above 9.0, the second mixed mode chromatography step can be performed in an aqueous buffered solution comprising 5 mM sodium phosphate, 170 mM sodium chloride, pH 7.5±0.2. Loading is performed in the same condition. As a further non-limiting example, e.g. for an protein of interest having a pI about 8.5 to about 9.5, the second mixed mode chromatography step can be performed in an aqueous buffered solution comprising 3 mM sodium phosphate, 470 mM sodium chloride, pH 7.5±0.2. Loading is performed in the same condition.

C.4. Alternative

The skilled person will understand, based on the present disclosure that he could also use as a first mixe mode step (steps (c)-(d)) a mixed mode chromatography support selected from the group consisting of hydroxy-based ligand and/or fluorapatite-based ligand and as a second mixe mode step (steps (e)-(f)) a mixed mode support selected from the group consisting of Capto-MMC, Capto-Adhere, Capto adhere Impress, MEP Hypercel and ESHMUNO HCX.

### D. Possible Additional Steps

D.1. Virus Inactivation

Optionally, the method according to the present invention comprises a step of virus inactivation. This step is preferably performed between the affinity chromatography step and the first mixed mode chromatography step. It is called step (b′). In order to inactivate viruses, the eluate recovered after affinity chromatography step (i.e. the eluate of step (b)) is adjusted with a concentrated acidic aqueous solution. The pH to be reached during adjustment is preferably in a range of or of about 3.0 to or to about 4.5, even preferably in a range of or of about 3.2 to or to about 4.0, such as 3.2, 3.3, 3.4, 3.5, 3.6, 3.7, 3.8, 3.9 or 4.0. The concentration of the salt in the acidic aqueous solution used for adjustment is at or at about 1.5 to or to about 2.5. Preferably, the concentration of the salt in the acidic aqueous solution is at or at about 1.7 to or to about 2.3, such as 1.7, 1.8, 1.9, 2.0, 2.1, 2.2, or 2.3 M. the preferred acidic aqueous solution is acetic acid. The resulting adjusted eluate is typically incubated for about 60±15 min.

At the end of the incubation, the material is then neutralized with a concentrated neutral aqueous solution. The pH to be reached during neutralization is preferably in a range of or of about 4.5 to or to about 6.5, should the neutralized sample be hold before step (c), even preferably in a range of or of about 4.8 to 5.6 such as 4.8, 4.9, 5.0, 5.1, 5.2, 5.3, 5.4, 5.5 or 5 5.6. Should the neutralized sample be directly used for step (c), pH to be reached during neutralization will be the same pH as the one that will be used for step (c), i.e. from 6.5 to 8.5. The concentration of the salt in the aqueous solution used for neutralization is at or at about 1.0 to or to about 2.5. Preferably, the concentration of the salt in the neutral aqueous solution is at or at about 1.0 to or to about 2.0, such as 1.0, 1.1, 1.2, 1.3, 1.4, 1.5, 1.6, 1.7, 1.8, 1.9, 2.0 M. The preferred neutral aqueous solution is Tris base.

D.2. Optional Filtration Steps

Various filtration steps can be added in the purification process. Such steps may be needed to further eliminate impurities but can also be used to concentrate the sample to be purified before the next chromatographic step or to change the buffer before the next chromatographic step.

For instance, in order to further reduce impurities of the eluate, or adjusted eluate, after step (b) or (b′), a filtration step can be performed just before the first mixed mode chromatography. This filtration step is preferably performed with a depth filter. Said step can be performed in line with the first mixed mode chromatography.

A filtration step, such as a depth filtration, can be included during the process. This step can for instance be added just before the the affinity chromatography or before the first mixed mode chromatography, as described in Example 2.

Tangential Flow Filtration (TFF) can also be performed during the purification procedure. For instance, should one wish to concentrate the flowthrough from step (d) before being loading on the second mixed mode chromatography, a TFF can be performed just before step (e). Such step, if any, is called step (d′). Such filtration step can be performed with the equilibration buffer that will be used for the second mixed mode chromatography. This will allow the flowthrough not only to be concentrated but also to be in such a condition to be ready for the next chromatographic step.

### EXAMPLES

### I. Cells, Cell Expansion and Cell Growth

“mAb1” is a humanized monoclonal antibody directed against a receptor found on the cell membrane. Its isoelectric point (p1) is about 9.20-9.40. mAb1 was produced in CHO-K1 cells.

“mAb2” is an IgG1 fusion protein, comprising one part directed against a membrane protein (IgG part, comprising an Fc domain) linked to a second part targeting a soluble immune protein. Its isoelectric point (pI) is about 6.6-8.0. It was expressed in CHO-S cells.

“mAb3” is a humanized monoclonal antibody directed against a receptor found on the cell membrane. Its isoelectric point (pI) is about 8.5-9.5. mAb3 was produced in CHO-S cells.

Cells were cultured in fed-batch culture. They were incubated at 36.5° C., 5% de CO₂, 90% humidity and shaken at 320 rpm. Each of the fed-batch culture lasted 14 days.

### II. Analytical Methods

Content in HCPs (ppm): HCPs level in ppm is calculated using the HCPs level determined in ng/mL divided by the mAb concentration determined by UV absorbance (mg/mL).

Content in aggregates (HMW)(expressed in % of protein concentration): the assessment was done by SE-HPLC, using a standard protocol.

Content in fragmented forms (LMW) (expressed in % of protein concentration): the assessment was done by CE-SDS, using a standard protocol.

### Example 1—MAb1 Purified according to a Standard Process

The full purification process was performed at room temperature (15-25° C.) except for the load step of the Protein A step, as the clarified harvest was stored at 2-8° C. before purification.

MAb1 was purified according to standard purification steps including “protein A chromatography” followed by a first “ion exchange chromatography” (IEX) in bind elute followed by a second IEX in flowthrough (also called polishing step).

Using said standard process, the following results were obtained:

| | Post | Post | Post | Total |
|---|---|---|---|---|
| | Protein | first | Second | purification |
| Impurities | A | IEX | IEX | factor |
| HCPs | 250 ppm | 50 ppm | 5 ppm | 50 |
| HMW | 1% | 0.6% | 0.6% | 1.7 |
| LMW | 2.8% | 3.1% | 3% | 0.9 |

### Example 2—MAb1 Purified according to the Process of the Invention

The full purification process was performed at room temperature (15-25° C.) except for the load step of the Protein A step, as the clarified harvest was stored at 2-8° C. before purification. The new process, according to the invention, had been used to improve the purification scheme for mAb1. The main steps for this new process were:
Protein A chromatography (PUP),
Mixed mode chromatography 1 (MM1),
Mixed mode chromatography 2 (MM2).

Protein A Step

Protein a step was performed on a Prosep Ultra Plus® resin (Merck Millipore), with a target bed height of 20±2 cm. This step was performed under the following conditions:
1. Equilibration: at least (≥)5 bed volume (BV) of an aqueous solution comprising 25 mM NaPI (sodium phosphate)+150 mM NaCl, pH 7.0. At the end of equilibration, the pH and conductivity of the effluent were checked. They should meet the recommendations of pH and conductivity of 7.0±0.2 and 18±1 mS/cm, respectively, before loading could start.
2. Load: clarified harvest at a maximum capacity of about 35-40 g mAb1/L of packed bed, at a temperature of 2-25° C.
3. Wash I: ≥5 BV of a solution comprising 55 mM sodium Acetate, 1.5M NaCl, pH 5.5.
4. Wash II: ≥3 BV of a solution comprising 25 mM NaPI+150 mM NaCl, pH 7.0.
5. Elution: with 55 mM acetic acid pH3.2. The eluate peak was collected as soon as the absorbance at 280 nm reaches 25 mAU/mm of UV cell path and the collection was stopped as soon as the absorbance at 280 nm is back at 25 mAU/mm of UV cell path. The eluate volume should be less than 4 BV.

Virus Inactivation at Low pH

The Protein A eluate was adjusted to pH 3.5±0.2 by addition of 2M acetic acid solution under stirring. Once the target pH was reached, the agitation was stopped and the acidified eluate was incubated for 60±15 min. At the end of the incubation, the material was neutralized to pH 5.2±0.2 by addition of 2M Tris Base solution under stirring. The resulting eluate (neutralized eluate) can be stored at least 3 months at 2-8° C.

Mixed Mode Chromatography 1

The neutralized eluate was adjusted to pH 8.0±0.2 with 2M Tris and its conductivity was increased to 15.0±0.5 mS/cm with 3M NaCl. This adjusted eluate was then submitted to depth filtration in line with mixed mode chromatography on Capto Adhere® (GE Healthcare) as follow:
1. The depth filter (Millistack Pod from Merck Millipore) was connected to the purification system in front of the chromatography column.
2. Pre equilibration of the resin: ≥3 BV of 500 mM NaPI, pH 7.5
3. Equilibration of the resin: ≥6 BV of 40 mM NaPI, 93 mM NaCl, pH 8.0.
4. Loading the adjusted eluate at a capacity of 100 g/L of mAb1/L of packed resin. Collection of the flowthrough started as soon as the absorbance at 280 nm reaches 12.5 mAU/mm of UV cell path.
5. Wash (=push): 4 BV of 40 mM NaPI, 93 mM NaCl, pH 8.0. Collection of the flowthrough containing the purified mAb1 was then stopped.

Mixed Mode Chromatography 2

Before being further purified in the mixed mode chromatography 2, the flowthrough from mixed mode chromatography 1 was concentrated via TFF, on a Pellicon 3 Ultracel® 30 kDa membrane (Merck Millipore). This step allowed also to exchange the buffer into conditions suitable for the load of the fluorapatite chromatography step, on a CFT Ceramic Fluorapatite® Type II (40 um) (Bio-Rad). The TFF step was performed as follow:
1. Equilibration of the filter (comprising both retentate and permeate lines): 5 mM NaPO4, 170 mM NaCl, pH 7.5 buffer.
2. Loading the flowthrough from mixed mode chromatography 1 at ≤500 g mAb1/m²
3. Diafilter with ≥9 DV of the same buffer as for equilibrium
4. Recover the retentate containing the purified mAb1.

The mixed mode chromatography 2 step was performed as follow:
1. Pre equilibration: ≥3 BV of 0.5M NaPI, pH 7.50.
2. Equilibration: ≥5 BV 5 mM NaPI, 170 mM NaCl, pH7.5
3. Loading the TFF retentate at a capacity ≤60 g mAb1/L of packed resin. Collection of the flow through started as soon as the absorbance at 280 nm reaches 12.5 mAU/mm of UV cell path.
4. Wash (=push): ≥6 BV with 5 mM NaPI, 170 mM NaCl, pH7.5. Collection of the flowthrough containing the purified mAb1 was then stopped.

Using said new process, the following results were obtained:

| | | | | Total |
|---|---|---|---|---|
| | Post | Post | Post | purification |
| Impurities | PUP | MM1 | MM2 | Factor |
| HCPs | 874 ppm | 36.7 ppm | 25.1 ppm | 35 |
| HMW | 2.4% | 0.5% | 0.2% | 12 |
| LMW | 2.7% | 2.3% | 1.4% | 1.9 |

### Example 3—Mab2 Purified according to a Standard Process

The full purification process was performed at room temperature (15-25° C.) except for the load step of the Protein A step, as the clarified harvest is usually stored at cold temperature (i.e. at 2-8° C.). Mab2 was purified according to standard purification steps including “protein A chromatography” followed by a first IEX in flowthrough followed by a second IEX in bind elute.

Using said standard process, the following results were obtain:

| | Total purification |
|---|---|
| Impurities | Factor |
| HMW | 1.9 |

### Example 4—Mab2 Purified according to the Process of the Invention

The full purification process was performed at a temperature between 20 and 23° C., except for the load step of the Protein A step, as the clarified harvest is usually stored at cold temperature (i.e. at 2-8° C.). The main steps for this new process were similar to example 2. Some amendments have been made to fit to the pI of mAb2:

At Mixed Mode Chromatography 1 Level

The neutralized eluate was dialysed to reach a pH 7.1±0.2 and 33±0.5 mS/cm of conductivity. This adjusted eluate was then submitted to mixed mode chromatography on Capto Adhere® (GE Healthcare) as in example 2. Beside:
1. Equilibration of the resin: ≥6 BV of 40 mM NaPI, 340 mM NaCl, pH 7.1.
2. Loading of the dialysed solution at a capacity of 100 g/L of mAb2/L of packed resin. Collection of the flow through started as soon as the loading step begun.
3. Wash (=push): ≥4 BV of 40 mM NaPI, 340 mM NaCl, pH 7.1. Collection of the flowthrough containing the purified mAb2 was stopped when the absorbance at 280 nm decrease under 100 mAU/mm of UV cell path.

At Mixed Mode Chromatography 2 Level

Before being further purified in the mixed mode chromatography 2, the flowthrough buffer is exchange into conditions suitable for the load of the fluoroapatite chromatography step, on a CFT Ceramic Fluoroapatite® Type II (40 um) (Bio-Rad).

The mixed mode chromatography 2 step was performed as follow:
1. Pre equilibration: ≥5 BV of 0.5 M NaPI, pH 7.50.
2. Equilibration: ≥15 BV 3 mM NaPI, 420 mM NaCl, pH7.5
3. Loading of the dialysed solution at a capacity ≤60 g mAb2/L of packed resin. The collection of the flow through start as soon as the loading step begin.
4. Wash (=push): ≥6 BV with 3 mM NaPI, 420 mM NaCl, pH7.5. Stop collection of the flowthrough containing the purified mAb2 when the absorbance at 280 nm decrease under 100 mAU/mm of UV cell path.

Using said new process, the following results were obtained:

| | Total purification |
|---|---|
| Impurities | Factor |
| HMW | 6.2 |

### Example 5—Mab3 Purified according to a Standard Process

The full purification process was performed at room temperature (15-25° C.) except for the load step of the Protein A step, as the clarified harvest is usually stored at cold temperature (i.e. at 2-8° C.). Mab3 was purified according to example 3.

Using said standard process, the following results were obtain:

| | Total purification |
|---|---|
| Impurities | Factor |
| HMW | 0.7 |
| LMW | 0.9 |

### Example 6—Mab3 Purified according to the Process of the Invention

The full purification process was performed at a temperature between 20 and 23° C., except for the load step of the Protein A step, as the clarified harvest is usually stored at cold temperature (i.e. at 2-8° C.). The main steps for this new process were similar to example 4. Some amendments have been made to fit to the pI of mAb3:

At Mixed Mode Chromatography 1 Level

The neutralized eluate was dialysed to reach a pH 7.3±0.2 and 46±0.5 mS/cm of conductivity. This adjusted eluate was then submitted to mixed mode chromatography on Capto Adhere® (from GE Healthcare) as in example 4. Beside:
1. Equilibration of the resin: ≥6 BV of 40 mM NaPI, 470 mM NaCl, pH 7.3.
2. Wash (=push): ≥1 BV of 40 mM NaPI, 470 mM NaCl, pH 7.3.

At Mixed Mode Chromatography 2 Level

Before being further purified in the mixed mode chromatography 2, the flowthrough buffer was exchange into conditions suitable for the load of the fluoroapatite chromatography step, on a CFT Ceramic Fluoroapatite@ Type II (40 um) (from Bio-Rad). The mixed mode chromatography 2 step was performed as in example 4.

Using said new process, the following results were obtained:

| | Total purification |
|---|---|
| Impurities | Factor |
| HMW | 4.3 |
| LMW | 1.2 |

### Conclusion

It was found by the inventors that using the process according to the present invention (as described in examples 2, 4 or 6 for instance), the purification of various antibodies and Fc-fusion proteins was improved compared to a standard process (as described in examples 1, 3 or 5 for instance). In particular it was possibly to decrease even more the quantity of impurities such as aggregates (HMW content) and fragments (LMW content), while keeping HCPs in acceptable ranges (data not shown).

### REFERENCES

[1] Davis et al., 2010, Protein Eng Des Sel 23: 195-202

[2] U.S. Pat. No. 8,871,912

[3] Sambrook et al., 1989 and updates, Molecular Cloning: A Laboratory Manual, Cold Spring Laboratory Press.

[4] Ausubel et al., 1988 and updates, Current Protocols in Molecular Biology, eds. Wiley & Sons, New York.

[5] Remington's Pharmaceutical Sciences, 1995, 18th ed., Mack Publishing Company, Easton, Pa.

[6] Horenstein et al., 2003, Journal of Immunological Methods 275:99-112.

1-14. (canceled)

15. A method of purifying a protein from a sample containing the protein and impurities, wherein the method comprises the following steps:
(a) contacting the sample containing the protein and the impurities with a protein A chromatography material under conditions such that the protein binds to the chromatography material and at least a portion of the impurities does not bind to the chromatography material;
(b) eluting the protein from the Protein A chromatography material, in order to obtain an eluate;
(c) loading the eluate of step (b) onto a first mixed mode chromatography material under conditions such that the protein does not bind to the chromatography material and at least a portion of the remaining impurities binds to the chromatography material;
(d) recovering the flowthrough containing the protein under conditions such that said recovered flowthrough contains a lower level of impurities than the eluate of step (b),
(e) loading the recovered flowthrough containing the protein of step (d) onto a second mixed mode chromatography material under conditions such that the protein does not bind to the chromatography material and at least a portion of the remaining impurities binds to the chromatography material; and
(f) recovering the flowthrough containing the protein under conditions such that said recovered flowthrough contains a lower level of impurities than the recovered flowthrough of step (d).

16. The method according to claim 15, wherein the protein is an Fc fusion protein or an antibody.

17. The method according to claim 15, wherein the protein has been produced in recombinant mammalian cells.

18. The method according to claim 15, wherein the mixed mode chromatography material of step (c) or (e) present a combination of two or more of the following functionalities: cation exchange, anion exchange, hydrophobic interaction, hydrophilic interaction, hydrogen bonding, pi-pi bonding and metal affinity.

19. The method according to claim 15, wherein the mixed mode chromatography material of step (c) is selected from the group consisting of Capto-MMC and Capto-Adhere and the mixed mode chromatography material of step (e) is selected from the group consisting of hydroxyapatite-based ligand, hydroxyfluorapatite-based ligand or fluorapatite-based ligand.

20. The method according to claim 19, wherein the mixed mode chromatography material of step (e) is a fluorapatite ligand of CFT type I or CFT type II.

21. The method according to claim 15, wherein the sample, containing the protein, to be contacting with the Protein A chromatography material in step a) is in an aqueous solution.

22. The method according to claim 15, wherein the protein A chromatography material is equilibrated, before step (a), with an aqueous buffered solution comprises between 20 and 30 mM of sodium phosphate, a salt at a concentration between 100 and 200 mM and has a pH in the range of 6.5 to about 7.5.

23. The method according to claim 15, wherein the elution of step (b) is performed with an elution buffer comprising between 40 and 70 mM of acetic acid at a pH in the range of 3.0 to about 3.5.

24. The method according to claim 15, wherein the mixed mode chromatography material of step (c) is equilibrated, prior to loading of the eluate of step (b), with an aqueous buffered solution comprising between 30 and 50 mM of sodium phosphate, a salt at a concentration between 80 and 120 mM and a pH in the range of 7.5 to about 8.5.

25. The method according to claim 15, wherein the mixed mode chromatography material of step (e) is equilibrated, prior to loading recovered flowthrough of step (d), with an aqueous buffered solution comprising between 1 and 10 mM of sodium phosphate, optionally a salt at a concentration between 130 and 200 mM and a pH in the range of 7.0 to about 8.0.

26. The method according to claim 22, wherein the salt is sodium chloride.

27. The method according to claim 15, wherein the impurities comprise aggregates or fragments of the protein being purified or mixtures thereof, host cell proteins, endotoxins, viruses, nucleic acid molecules, lipids, polysaccharides, and any combinations thereof.

28. The method according to claim 24, wherein the salt is sodium chloride.

29. The method according to claim 25, wherein the salt is sodium chloride.

30. A method of obtaining a protein in a monomeric form, wherein the method comprises the following steps:
(a) contacting the sample containing the protein in monomeric form, aggregated form or fragmented form with a Protein A chromatography material under conditions such that the protein in monomeric form binds to the chromatography material and at least a portion of the aggregated forms and fragmented forms does not bind to the chromatography material;
(b) eluting the protein in monomeric form from the Protein A chromatography material, in order to obtain an eluate;
(c) loading the eluate of step (b) onto a first mixed mode chromatography material under conditions such that the protein in monomeric form does not bind to the chromatography material and at least a portion of the remaining aggregated forms and fragmented forms bind to the chromatography material;
(d) recovering the flowthrough containing the protein in monomeric form under conditions such that said recovered flowthrough contains a lower level of aggregated forms and fragmented forms than the eluate of step (b),
(e) loading the recovered flowthrough containing the protein in monomeric form of step (d) onto a second mixed mode chromatography material under conditions such that the protein in monomeric form does not bind to the chromatography material and at least a portion of the remaining aggregated forms and fragmented forms bind to the chromatography material; and
(f) recovering the flowthrough containing the protein in monomeric form under conditions such that said recovered flowthrough contains a lower level of aggregated forms and fragmented forms than the recovered flowthrough of step (d).
